(19) United States
(12) Patent Application Publication
(10) Pub. No.: US 2018/0098720 A1
(43) Pub. Date: Apr. 12, 2018

(54) A Method and Device for Conducting a Self-Administered Hearing Test

(71) Applicant: MEQ INC., Albany, CA (US)

(72) Inventors: Ofer RAZ, Albany, CA (US); Danny ARONSON, Tel Aviv (IL)

(73) Assignee: MEQ INC., Albany, CA (US)

(21) Appl. No.: 15/567,115

(22) PCT Filed: Apr. 12, 2016
(86) PCT No.: PCT/IL2016/000006

§ 371 Date: Oct. 17, 2017

Related U.S. Application Data
(60) Provisional application No. 62/149,066, filed on Apr. 17, 2015.

(51) Int. Cl. A61B 5/12; H04R 1/10

(52) U.S. Cl. CPC A61B 5/123; H04R 1/1008; H04R 1/1041; A61B 2560/0247; A61B 2562/0204; H04R 1/1091

(57) ABSTRACT

A set of headphones is provided, configured to enable a user of the set of headphones to conduct a self-administered hearing test. The set of headphones comprises: a sound generating module; a controller operative to control separately sounds reaching each one of the user's ears; at least one processor operative to process feedbacks received via a user interface coupled to the set of headphones, and wherein the at least one processor is operative to: allow uploading of executable instructions to enable performing steps in a self-administered hearing test; activate the controller and the sound generating module in accordance with the executable instructions, for conducting the self-administered hearing test by the user using the set of headphones; receive the feedbacks from the user while conducting the self-administered hearing test; and determine hearing capabilities associated with the user, based on results obtained from the self-administered hearing test.

### TECHNICAL FIELD

The present disclosure relates to the field of consumer electronics, and in particular to devices such as headphones for listening to audio signals, and to methods for using them.

### BACKGROUND

Headphones are a pair of small loudspeakers that are designed to be held close to a user's ears. Different types of headphones have different sound reproduction characteristics.

Headphones may be used both with fixed equipment such as CD or DVD payers, home theater, personal computers and with portable devices (e.g. digital audio/player/mp3 player, mobile phone, etc.).

Headphones are available with low or high impedance (typically measured at 1 kHz): Low-impedance headphones are in the range 16 to 32 ohms and high-impedance headphones are about 100-600 ohms. As the impedance of a pair of headphones increases, more voltage (at a given current) is required to drive it, and the loudness of the headphones for a given voltage decreases. The impedance of headphones is of concern because of the output limitations of amplifiers. A modern pair of headphones is driven by an amplifier, with lower impedance headphones presenting a larger load. Amplifiers are not ideal; they also have some output impedance that limits the amount of power they can provide. If output impedance is large compared to the impedance of the headphones, significantly higher distortion will occur. Therefore, lower impedance headphones will tend to be louder and more efficient, but will also demand a more capable amplifier. Higher impedance headphones will be more tolerant of amplifier limitations, but will produce less volume for a given output level.

Sensitivity is a measure of how effectively an earpiece converts an incoming electrical signal into an audible sound. It thus indicates how loud the headphones will be for a given electrical drive level. It can be measured in decibels of sound pressure level per milliwatt, or dB SPL/mW, which may be abbreviated to dB/mW. The sensitivity of a given pair of headphones is fixed and is usually between about 80 and 125 dB/mW.

Everyone uses headphones the same way as the others, but since hearing capabilities change from one person to the other, the audio signals to which the headphones' users are able to listen, change from one person to the other, depending primarily on one's hearing capabilities. The term “hearing range” usually describes the range of frequencies that can be heard by humans, though it can also refer to the range of levels. The human range is commonly given as 20 to 20,000 Hz, though there is considerable variation between different individuals, especially at high frequencies, and a gradual decline with age is considered normal. Sensitivity also varies with frequency, as shown by equal-loudness contours. Individual hearing range varies according to the general condition of a human's ears. The range shrinks during life, usually beginning at around the age of eight with the upper frequency limit being reduced. women typically experience a lesser degree of hearing loss than men, with a later onset. Men have approximately 5 to 10 dB greater loss in the upper frequencies by the age of 40. On top of that, there may also be substantial differences between one's hearing capabilities in the left and right ears.

In view of the above, it is clear that the listening experience of each user could be enhanced if the audio output to which he/she listens, fits that individual. To do that, it is preferable to conduct a hearing test to each such user in order to try and tailor a compensation for the degradation in the user's own hearing capabilities.

Traditionally, a hearing test is conducted in a clinical setting by a hearing health professional, such as an audiologist, who manually administers the hearing test. The hearing health professional controls an audiometer to produce a series of tones that each have a specific frequency and intensity. The term “intensity” as used herein refers to the amplitude of the tone and is usually stated in decibels (dB). The tones are then conducted through a transducer, such as earphones or ear inserts, to the patient in a quiet room or sound isolation booth. For each tone that is audible, the patient gestures or otherwise indicates that he has heard the tone. If the tone is not audible, the patient does not respond. The hearing health professional thereafter adjusts the intensity level of the tone in preset increments until the tone becomes audible to the patient. By repeating this process for several different tones and compiling the results, the hearing health professional is able to determine the extent of the hearing loss, if any.

Traditional audiometers and other hearing test equipment, however, can be awkward and difficult to use. For example, traditional audiometers typically require the hearing health professional to manually turn knobs and adjust dials. Test information such as frequency and intensity level must typically be read from needle gauge readouts.

Certain solutions were proposed in the art in order to facilitate the task of diagnostic testing of a patient's hearing. One such solution is described in U.S. Pat. No. 8,529,464 which teaches the use of a computer-assisted diagnostic testing of a patient's hearing. The computer-assisted diagnostic hearing test uses computer-based hearing test equipment that include a user-friendly multimedia interface for facilitating performance of the various hearing related tests.

U.S. Pat. No. 8,308,653 discloses a system for automated testing of a patient's hearing and a method for using it. The system has three main components, namely, a computer, a display screen, and at least one transducer. Other components of the system that may be present include a tympanometer, peripheral equipment (such as keyboard, mouse, printer) and paging system, that uses one or more pagers or other wireless mobile devices for alerting the operator. The mobile terminal preferably can display text messages for informing the operator of the nature of the alert.

U.S. Pat. No. 8,675,900 discloses a system for determining a user's own hearing threshold. The system includes a smartphone, a viewing screen of the smartphone, smartphone software, ear level hearing device, transmitter on the smartphone and receiver on the ear level device. The interface on the smartphone includes an automatic routine or buttons to vary frequency and amplitude of a frequency dependent sound presentation to the earpiece. The software installed on the smartphone sends wireless signals to the ear level device changing acoustic parameters in the listening device. The ear level device stores frequency/amplitude parameters of the hearing's thresholds and wirelessly delivers them to the smartphone. The smartphone uses the threshold data to derive the appropriate amplified acoustical signal (relative to the thresholds) to the user.

There is a problem to carry out a hearing test using audio signals that are played directly by a smartphone or any other non-calibrated device, as the user cannot pre-determine or control the level (volume) of the signals which are affected by the user's device volume setting, as well as by the hardware implemented in that specific smartphone. Additionally, the user cannot direct the sound only to one ear (channel). Furthermore, in phones and headphones used today, the sound typically leaks between the left and right channels.

Thus, it is common that hearing tests are done either in a controlled environment (such as a dedicated booth for example) with a trained audiologist or by using a portable device such as a smartphone. Obviously, the latter option is the one that will be used for carrying out a self-administered hearing test. However, there is an inherent problem associated with carrying out hearing tests where the tones heard by the user are played by a smartphone or by any other non-calibrated device. The reason being that when using such devices, it is impossible to determine or control the level (volume) of the signal as well as to ensure that the tone is heard by only one ear of the user (i.e. that the tone is delivered from the smartphone directly to one ear). In addition, as explained above, phones and headphones that are commonly used nowadays suffer from the disadvantage that the sound (tones) is likely to leak between the left and right channels.

In order to overcome these problems, while still being able to carry out a self-administered hearing test, the user would be required to purchase a separate calibration device (e.g., a coupler and sound level meter) and then manually calibrate the output of the smartphone software program. However, in addition to the drawbacks involved with the costs incurred by implementing such a solution, calibrating the tonal output may turn out to be a rather difficult task, especially for a person who is not trained in audiology. Furthermore, while some self-administered hearing tests attempt to measure and display threshold levels, the meaning and implications of such results can remain unclear to a user carrying out the test, who is unfamiliar with standard hearing profiles.

Accordingly, there is a need for further improvements to enable user to carry out self-administered hearing tests.

### SUMMARY OF THE DISCLOSURE

The disclosure may be summarized by referring to the appended claims.

It is an object of the present disclosure to provide a method to enable carrying out a self-administered hearing test by a user.

It is another object of the present disclosure to provide a method and a set of headphones that enable a user to conduct a self-administered hearing test using the headphones' set without being required to use any additional hardware other than the set of headphones itself.

It is another object of the disclosure to provide a method and a set of headphones that enable a user to conduct a self-administered hearing test at a substantially reduced time when compared to hearing tests known in the art.

It is another object of the present disclosure to provide a method to enable modifying audio signals in accordance with results obtain in a self-administered hearing test for determining the hearing capabilities of a user who is listening to the audio signals.

It is yet another object of the present disclosure to provide a new set of headphones for enabling a user to carry out a self-administered hearing test.

It is still another object of the present disclosure to provide a device which is capable of modifying audio signals in accordance with results obtained in a self-administered hearing test for a user who is listening to the audio signals via a given set of headphones.

Other objects of the present invention will become apparent from the following description.

According to a first embodiment of the present disclosure, there is provided a method for enabling a user of a set of headphones to conduct a self-administered hearing test, comprising:

providing a set of headphones;

providing a sound generating module;

providing a controller operative to control separately sounds reaching each of the user's ears;

providing at least one processor operative to process feedbacks obtained from the user of the set of headphones;

providing a user interface (e.g. a button) coupled to the set of headphones, which enables receiving feedbacks from the user;

loading executable instructions onto the at least one processor to enable performing steps in a self-administered hearing test;

activating the controller and the sound generating module in accordance with the executable instructions, for conducting the self-administered hearing test by the user using the set of headphones;

receiving feedbacks from the user while conducting the self-administered hearing test; and

determining hearing capabilities associated with the user, based on results obtained from the self-administered hearing test.

The term “set of headphones” as used herein throughout the specification and claims is used to denote typically a pair of small loudspeakers designed to be held in place close to a user's ears. They are also known as earspeakers, earphones, drivers, etc. Typically, there are three types of headphones—in-ear, on the ear and covering the entire ear. The in-ear versions are commonly known as earbuds or earphones. Headphones either have wires for connection to a signal source such as an audio amplifier, radio, CD player, portable media player, mobile phone, electronic musical instrument, etc., or have a wireless device, which is used to pick up audio signals without using a cable. Also, although this term typically relates to a pair of headphones it should be understood to encompass also other cases, e.g. a single headphone.

The term “hearing test” as used herein throughout the specification and claims is used to denote a test being carried out to determine the user's hearing capabilities at one or both of his/her ears. The hearing test may be conducted as two separated tests, each for a different user's ear, or as one test for determining the combined user's hearing capabilities, when both ears are being subjected simultaneously to the hearing test.

The term “hearing capabilities” as used herein throughout the specification and claims is used preferably but not exclusively to denote gaps that exist when audio tones are heard by a user who listens to these tones as opposed to the same audio tones as they should have been heard by the user, had he/she had a perfect hearing ability. The gap may be different for different frequencies (or for different range of frequencies) and may also vary between the user's ears. This gap may then be used to enable affecting certain modifications of various musical tones played via the user's set of headphones, in order to decrease these gaps (which characterize the specific user), thereby enhancing his/her hearing experience when listening to a musical creation while using the set of headphones of the present invention.

The terms “a sound generating module” and “a controller” as used herein throughout the specification and claims, should be understood to also encompass any other means suitable for generating sounds in a controlled way, e.g. by using the processor. Thus, all such other means whether in a form of a single means or a combination of means that are used to generate an outcome that is equivalent to that of using the sound generating module and/or controller, i.e. means that are used in a way that they enable generating sounds in a controlled way, are encompassed by the present invention.

One of the steps of the method provided by the present invention is “loading executable instructions onto the at least one processor to enable performing steps in a self-administered hearing test”. As will be appreciated by those skilled in the art, this step should be understood as being carried out either during the manufacturing process of the set of headphones, or be carried out by uploading the executable instructions (e.g. a code) at any later stage. All these options should be understood as being encompassed by the present invention.

By still another embodiment, the one or more hearing tests comprises testing the user's hearing capabilities at frequencies which are above 3000 Hz (e.g. up to about 14 KHz).

According to another embodiment, the sound generating module, the controller and the at least one processor are all comprised within the set of headphones.

By yet another embodiment, the method further comprises a step of processing sounds that are generated by the sound generating module, and automatically adjusting the volume and/or the amplitude of the audio signals generated by the sound generating module.

In accordance with another embodiment, the method further comprises a step of selectively controlling conveyance of audio signals towards one of the user's ears by the controller.

According to still another embodiment, the method further comprises a step of preventing audio leakage of sounds from one side of the set of headphones to the other.

By yet another embodiment, the method provided further comprises a step of storing information concerning the hearing capabilities associated with the user retrieved from the results obtained in the self-administered hearing test (e.g. storing the information in a memory included within the set of headphones). In an alternative, the information may be remotely stored (e.g. cloud storage) and when required, retrieved therefrom (e.g. wirelessly). Also, it should be understood that cases wherein the stored information comprises information that had been derived based on the retrieved information, are also included within the scope of the present disclosure and hence are encompassed by the present invention. In addition, the method may further comprise a step of modifying input audio signals in accordance with the stored information, thereby enabling the user to listen to modified audio signals, being the input audio signals after they have been changed into a form that takes into account the user's hearing capabilities.

In accordance with another embodiment, the processor is further operative to determine, based on the hearing test results, for at least one of the user's ears, at least one frequency at which the input audio signal will be modified, and the modification (e.g. change of amplitude, change of level of the compensating signal, change frequency etc.) that will be carried out to at least a portion of the audio signal associated with that at least one frequency.

By yet another embodiment, based on the test results obtained, the modification of the input audio signal is carried out at at least one frequency that is different from the one or more frequencies at which the test results were obtained.

In accordance with another embodiment, the method further comprises a step of determining for at least one of the user's ears, at least one range of frequencies at which the input audio signals will be modified, and the modification(s) that will be carried out at that range of frequencies.

According to another embodiment, the step of determining hearing capabilities associated with the user, comprises adjusting the hearing capabilities associated with the user, based on one or more ambient noises (e.g. based on pre-defined ambient noise(s)).

By yet another embodiment, the step of determining hearing capabilities associated with the user, comprises establishing a reference model for the hearing capabilities of the user, based on results obtained from his/her self-administered hearing test.

In accordance with still another embodiment, the method further comprises a step of providing at least one microphone for detecting current ambient noise(s) and a step wherein the determination of hearing capabilities associated with the user is further based on the current ambient noise(s) as detected by the at least one microphone.

According to another embodiment the method provided further comprises a step of generating a constant tone at a pre-defined frequency spectrum and a pre-defined amplitude range (a pink tone), for use while determining hearing capabilities associated with the user.

By yet another embodiment the method provided comprises the steps of:
(i) selecting a first frequency band and a respective initial intensity for generating audio tones at that first frequency band;
(ii) determining a user's hearing threshold associated with the selected first frequency band, by generating audio tones at the selected first frequency band at different intensities, starting from the respective initial intensity selected for first frequency band, and changing the intensity of the following audio tones generated, until a user's respective hearing threshold for the first frequency band is determined based on one or more feedbacks received from the user;
(iii) selecting another frequency band and a respective initial intensity for generating audio tones at that other frequency band, wherein the respective initial intensity for generating audio tones at that other frequency band is higher than the user's hearing threshold determined for an already tested frequency band (e.g. the user's hearing threshold determined for the first frequency band);
(iv) determining a hearing threshold of the user in the selected other frequency band, by generating audio tones at the selected other frequency band at different intensities, starting from the respective initial intensity associated with that other frequency band, and changing the intensity of the following audio tones until a user's respective hearing threshold for the other frequency band is determined, based on one or more feedbacks received from the user; and
(v) repeating steps (iii) and (iv) for different frequency bands, until completing the self-administered hearing test.

According to another embodiment, the first frequency band is associated with a frequency that is higher than at least one of the other frequency bands and is lower than that at least one of the other frequency bands.

By still another embodiment, the respective initial intensity for generating audio tones at one of the other frequency bands is higher than the user's hearing threshold determined for an already tested frequency band, by a pre-defined difference.

In accordance with another embodiment, the pre-defined difference has a constant value for all other frequency bands tested in the self-administered hearing test.

In accordance with another aspect of the present disclosure, there is provided a set of headphones configured to enable a user of the set of headphones to conduct a self-administered hearing test, and comprising:

a sound generating module;

a controller operative to control separately sounds reaching each one of the user's ears;

at least one processor operative to process feedbacks received via a user interface coupled to the set of headphones, and wherein the at least one processor is operative to;

allow uploading of executable instructions to enable performing steps in a self-administered hearing test;

activate the controller and the sound generating module in accordance with the executable instructions, for conducting the self-administered hearing test by the user using the set of headphones;

receive feedbacks from the user while conducting the self-administered hearing test; and

determine hearing capabilities associated with the user, based on results obtained from the self-administered hearing test.

According to another embodiment of this aspect the at least one processor is further configured to process sounds (i.e. audio signals) generated by the sound generating module, and to automatically adjust their volume and/or amplitude.

In accordance with another embodiment, the controller is operative to control selective conveyance of sounds towards one of the user's ears.

By yet another embodiment, the controller is operative to prevent audio leakage of sounds from one side of the set of headphones to the other.

According to still another embodiment, the set of headphones further comprises a memory for storing information concerning the hearing capabilities associated with the user, retrieved from the results obtained in the self-administered hearing test. Preferably, the at least one processor is further operative to modify input audio signals in accordance with the stored information, thereby enabling the user to listen to modified audio signals, being incoming audio signals after they have been changed into a form that takes into account the user's hearing capabilities.

By still another embodiment, the at least one processor is configured to determine hearing capabilities associated with the user, based on pre-defined ambient noise(s).

In accordance with another embodiment, the at least one processor is configured to determine hearing capabilities associated with the user, by establishing a reference model for the hearing capabilities of the user, based on the results obtained from his/her self-administered hearing test.

According to another embodiment of this aspect of the present disclosure, the set of headphones further comprises at least one microphone for detecting current ambient noise(s) and the at least one processor is configured to determine hearing capabilities associated with the user while taking into account the current ambient noise(s) as detected by the at least one microphone.

By yet another embodiment, the sound generating module is further configured to generate a constant tone at a pre-defined frequency and a pre-defined amplitude, for use by the at least one processor in determining hearing capabilities associated with the user.

According to another aspect of the disclosure, there is provided a computer program product encoding a computer program stored on a non-transitory computer-readable medium for executing a set of instructions by one or more computer processors for establishing a process for carrying out the steps of:

uploading executable instructions to enable performing a self-administered hearing test;

activating a controller and a sound generating module comprised within a set of headphones in accordance with the executable instructions, for conducting the self-administered hearing test by a user using the set of headphones;

receiving feedbacks from the user while conducting the self-administered hearing test; and

determining hearing capabilities associated with the user, based on results obtained from the self-administered hearing test.

By yet another embodiment of this aspect of the invention, the process further comprises the steps of:
(i) selecting a first frequency band and a respective initial intensity for generating audio tones at the first frequency band;
(ii) determining a user's hearing threshold associated with the selected first frequency band, by generating audio tones at the selected first frequency band at different intensities, starting from the respective initial intensity selected for first frequency band, and changing intensity of the following audio tones generated, until a user's respective hearing threshold for the first frequency band is determined based on one or more feedbacks received from the user;
(iii) selecting another frequency band and a respective initial intensity for generating audio tones at that other frequency band, wherein the respective initial intensity for generating audio tones at that other frequency band is higher than the user's hearing threshold determined for an already tested frequency band;
(iv) determining a hearing threshold of the user in the selected other frequency band, by generating audio tones at the selected other frequency band at different intensities, starting from a respective initial intensity associated with the other frequency band, and changing the intensity of the following audio tones until a user's respective hearing threshold for the other frequency band is determined, based on one or more feedbacks received from the user; and
(v) repeating steps (iii) and (iv) for different frequency bands, until completing the self-administered hearing test.

### BRIEF DESCRIPTION OF THE DRAWINGS

For a more complete understanding of the present invention, reference is now made to the following detailed description taken in conjunction with the accompanying drawings wherein:

FIG. 1—is a flow diagram exemplifying a method carried out in accordance with an embodiment of the present invention;

FIG. 2—is an example of the results of hearing tests performed on three different individuals; and

FIG. 3—is an example of the results of a hearing test performed on an individual according to an embodiment of the invention.

### DETAILED DESCRIPTION

In this disclosure, the term “comprising” is intended to have an open-ended meaning so that when a first element is stated as comprising a second element, the first element may also include one or more other elements that are not necessarily identified or described herein, or recited in the claims.

In the following description, for the purposes of explanation, numerous specific details are set forth in order to provide a better understanding of the present invention by way of examples. It should be apparent, however, that the present invention may be practiced without these specific details.

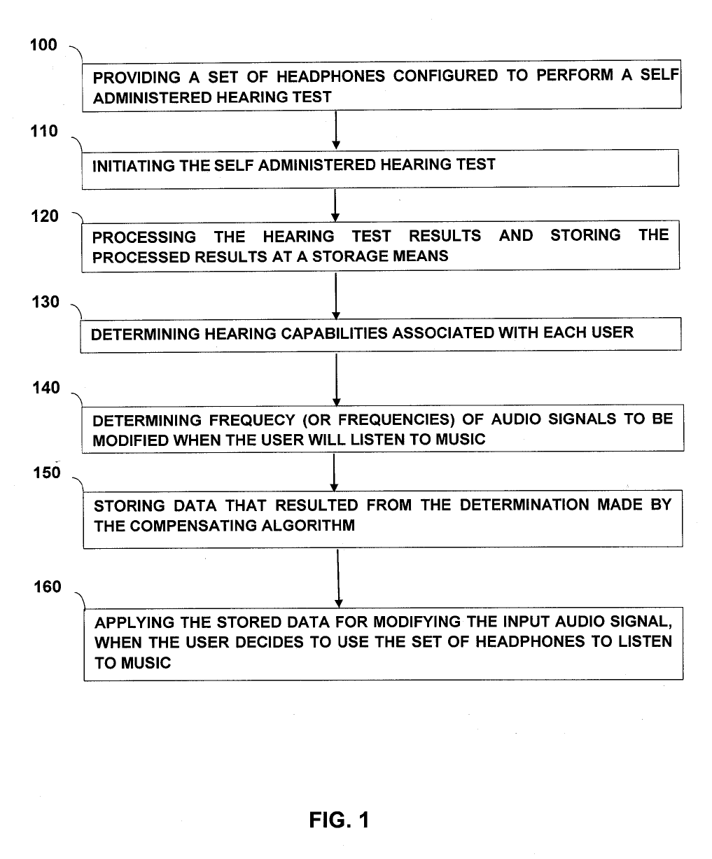

FIG. 1 is a flow diagram being a non-limiting example of a method carried out in accordance with an embodiment of the present invention.

In step 100 a set of headphones is provided, The set of headphones comprises a pair of earphones, a sound generating module, a controller operative to control separately sounds reaching each one of the pair of earphones, and a processor uploaded with an Android/iPhone/iPad/Laptop software application enabling to prompt users to go through a comprehensive, self-administered hearing test. These tests differ from standard hearing tests as they are designed to test the user hearing capabilities that pertain to the comprehension and enjoyment of music, and are thus different from conventional tests that focus solely on frequencies that make speech more comprehensible for those experiencing hearing loss. In addition, the user is provided with an interface coupled to the set of headphones, to enable receiving feedbacks from the user.

When the test is initiated (either by the user or by others, as the case may be), step 110, the controller is activated as well as the sound generating module in accordance with the executable instructions of the uploaded software, for conducting the self-administered hearing test by the user using the set of headphones.

The hearing tests of three users were performed for each ear of each of the users, generating two distinct audiograms for each user, the user's left and right ears.

The term “audiogram” as used herein is used to denote a graph that shows the audible threshold for frequencies as measured by an audiometer. The Y axis represents intensity measured in decibels while the X axis represents frequency measured in Hertz. The threshold of hearing is plotted relative to a standardized curve that represents normal hearing, in dB.

For humans, normal hearing is between −10 dB and 15 dB, although 0 dB from 250 Hz to 8 kHz is deemed to be ‘average’ normal hearing. Typically, hearing thresholds of humans are found by using hearing tests which involve different tones being presented at a specific frequency (pitch) and intensity (loudness). When the person hears the sound they press a button so that the testing means recognizes that they have heard it. The lowest intensity sound they can hear is recorded.

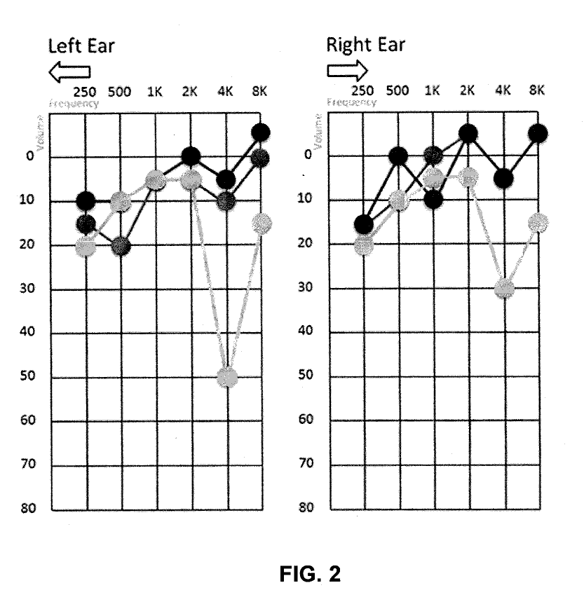

The actual audiograms that resulted from the self-administered hearing tests performed by each of these three users (for their left ears and right ears), are presented in FIG. 2. As may be seen in this Fig., there are substantial differences, for example between the hearing capabilities in both ears of, individual No. 3 as compared with the hearing capabilities of individuals 1 and 2, and particularly in the range of 2000 to 8000 Hz.

The results obtained in these self-administered hearing tests were processed and stored by the processor at a storage, while conducting the self-administered hearing test (step 120).

Next, the processor determined the hearing capabilities associated with the respective user based on results obtained while his self-administered hearing test was carried out (step 130).

By using a compensating algorithm, stored at the processor, which may include executable instructions to carry out an equalization process, the processor determines frequency ranges at which audio signals should be modified (step 140) by applying the appropriate compensation as required for the respective individual. In other words, the compensating algorithm uses predetermined equalizing (EQ) bands and curves to determine the compensation that will be provided to each of the user's ears. Typically, the compensation (e.g. the frequency ranges in which the modification will be carried out and/or the magnitude of compensation) that will be provided to the audio signal(s) reaching the user's left ear will be different from the compensation that will be provided to the audio signal(s) reaching the user's right ear, based on the resulting audiograms.

It should be noted that determining the required compensation based on the self-administered hearing test is a complex process, which takes into account different frequencies and bands (in terms of the range of frequencies affected) and does not create a simple “mirror” image of the users' audiogram. Instead, it is preferably used to generate a smooth and enjoyable equalized curve for each ear. For example, if a user has a 3 db drop at around 500 Hz, it might be more musically pleasing to enhance the frequencies around 500 Hz by 2.5 db through a broadband curve extending from 450 Hz to 570 Hz, rather than just bumping the 500 Hz drop by 3 db. Another example could be to reduce the level of few frequencies around the 500 Hz frequency instead of bumping the signal at exactly the 500 Hz frequency.

Next, in step 150, data resulting from the determination made by the compensation algorithm, are stored (at the storage means, e.g. as a firmware at the DSP comprised within the headphones), and when the user decides to use the headphones to listen to music, the data resulting from the compensation algorithm that has already been stored, is provided to two customized equalizers (for the L/R ears) in such a way that the audio signals may be modified in each of the two channels, respectively.

Optionally, but not necessarily, after completing the compensation determination process described above for both ears of the user, the user is able to do a further test where he/she would listen to a piece of music with and without the modification, followed by the user indicating whether a different compensation should be applied. Once the user has indicated that the compensation results are satisfactory, the equalization data (i.e. Frequency, Volume and “Q” value) are forwarded to the processor comprised within the headphones themselves, for storing the customized equalization data in the headphones.

The value of parameter “Q” referred to above, relates to the angle and scope of the gain given for each frequency band. It may be very sharp and bell shaped, thus affecting only a rather narrow range of frequencies, or on the other hand it may be quite flat and large, thus affecting a larger range of frequencies.

When the user decides to listen to music (step 160) through his own headphones, the audio signals are modified in conformity with the compensation determined for each of the user's ears, regardless of the device being used as the source of the music.

Although the compensation process described in this example relates to compensating each ear separately, it should be clear that the present invention also encompasses cases where the compensation is made for one ear only, or where there is essentially the same compensation for both ears of the users. At the latter case, the hearing test may be one test where both ears are tested simultaneously to receive a combined result, or that the ears are tested separately and the results obtained for each of the two ears are combined while determining the compensation that will be applied while modifying the audio signals.

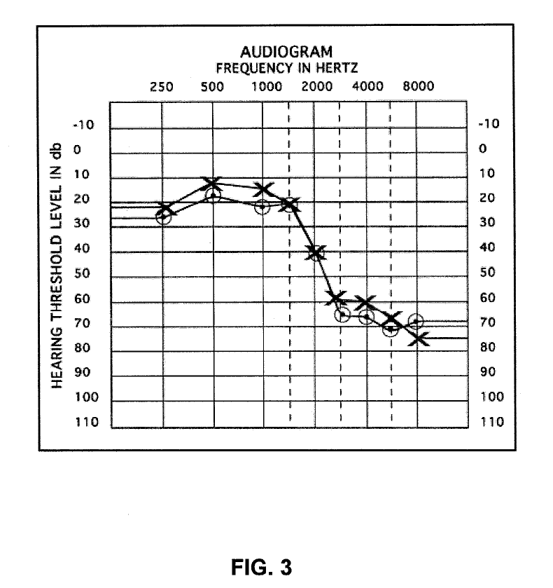

FIG. 3 is an example illustrating two audiograms (for the left ear and the right ear) obtained in a hearing test performed for a certain individual in accordance with an embodiment of the invention.

As known in the art, the time required to carry out a hearing test for an individual is rather a considerable barrier for implementing this type of a test, as the vast majority of the users/customers want the hearing test to be fast, and not spend a considerable amount of time to carry it out. There are different methods known in the art for shortening the time period required for performing a hearing test. Typically, these methods involve performing a less detailed hearing test. For example, the hearing test may include fewer bands (frequencies). Obviously, although these methods yield certain results, the results obtained are less accurate, hence they are unreliable for incorporating them in the modification of audio signals that would be based on these results.

The inventors of the present invention found that for each frequency band there is a zero point with represents a threshold of a tone intensity associated with a perfect hearing at that frequency band. This zero point may be different for each frequency band and reflects the biology and physics of the user's hearing. Usually a hearing test would start by generating an audio tone for the user, that has the intensity of the zero point and the hearing test would be concluded when the user is able to hear the tone at an increased intensity.

As people get older, their hearing capabilities deteriorate and they would begin hearing the audio tones at a much higher intensity (higher decibels) than the zero point. In most of the cases, when plotting the results in a graph, the shape of the graph would be a shape of a curve with the highest point (i.e. closer to the zero) at a frequency band around 500 Hz.

Preferably, in order to reduce the hearing testing time, the solution provided by the embodiment of the present invention allows changing the order of the hearing test. In other words, the self-administered hearing test begins at about the center of the graph, estimates the highest point of the individual's audiogram and uses that as a baseline for the hearing test starting point.

As the self-administered hearing test progresses, it becomes possible to predict the intensity at which the hearing test for the next band that will be tested should begin, based on whether higher or lower frequency bands are about to be tested.

By following this method, the inventors found that they were able to reduce the hearing test time less than a half of the original testing time, while still maintaining the quality of the results obtained in such a hearing test.

The following embodiment describes a method to expedite the self-administered hearing test, which comprises the steps of:

selecting a first frequency band and a respective initial intensity for generating audio tones at said first frequency band;
(i) determining a user's hearing threshold associated with the selected first frequency band, by generating audio tones at the selected first frequency band at different intensities, starting from the respective initial intensity selected for first frequency band, and changing intensity of the following audio tones generated, until a user's respective hearing threshold for the first frequency band is determined based on one or more feedbacks received from the user;
(ii) selecting another frequency band and a respective initial intensity for generating audio tones at that other frequency band, wherein the respective initial intensity for generating audio tones at that other frequency band is higher than the user's hearing threshold determined for an already tested frequency band;
(iii) determining a hearing threshold of the user in the selected other frequency band, by generating audio tones at the selected other frequency band at different intensities, starting from a respective initial intensity associated with the other frequency band, and changing the intensity of the following audio tones until a user's respective hearing threshold for the other frequency band is determined, based on one or more feedbacks received from the user; and
(iv) repeating steps (iii) and (iv) for different frequency bands, until completing the self-administered hearing test.

Let us now consider a specific example which is illustrated in FIG. 3, in order to demonstrate this embodiment of the present invention.

The hearing test began at a frequency band of 500 Hz. The intensity of the hearing threshold for this frequency band (500) was determined after increasing the intensity of the audio signals being played to the user taking the hearing test, starting from −10 dB, and receiving his feedback. Once the intensity of the hearing threshold for this frequency band was determined, another frequency band was selected. In this case, the frequency band of 250 Hz. The intensity of the hearing threshold for this frequency band (250) was determined after increasing the intensity of the audio signals being played to the user taking the hearing test, but this time, starting from +5 dB higher than the intensity of the hearing threshold determined for the frequency band of 500 Hz. In other words, for testing the 250 Hz frequency band, an initial intensity around +15 dB was used, instead of starting from −10 dB, thus saving five steps of the test (assuming that the hearing test is conducted in 5 dB steps). Next, another frequency band was tested, where this time the tested frequency was a higher one than the initial frequency, 1000 Hz. Again, the intensity of the hearing threshold for this frequency band (1000) was determined after increasing the intensity of the audio signals being played to the user taking the hearing test, but this time, starting from +5 dB higher than the intensity of the hearing threshold determined for the frequency band of 500 Hz.

Next, the test continued by generating audio tones at a frequency band of 2000 Hz. In this case, the intensity of the hearing threshold for this frequency band (2000) was determined as before, by increasing the intensity of the audio signals being played to the user taking the hearing test, but this time, starting from +5 dB higher than the intensity of the hearing threshold determined for the frequency band of 1000 Hz.

The process continued in a similar way, mutatis mutandis, for other frequency bands, until the hearing test was concluded and its results were stored for future use. For example, to enable modifying audio signals that will be played to the user, in accordance with his/her hearing capabilities.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

The present invention has been described using detailed descriptions of embodiments thereof that are provided by way of example and are not intended to limit the scope of the invention in any way. The described embodiments comprise different features, not all of which are required in all embodiments of the invention. Some embodiments of the present invention utilize only some of the features or possible combinations of the features. For example, although the description above relates to carrying out the method provided by components comprised within the set of headphones, still, it should be appreciated that the method may be exercised by having an intermediate device comprising the DSP described above which will be fitted between any headphone jack and the headphones themselves (any set of headphones). This will allow users to improve their sound listening experience by implementing the method described herein, without having to change their set of headphones, or without having to buy any particular set of headphones.

Variations of embodiments of the present invention that are described and embodiments of the present invention comprising different combinations of features noted in the described embodiments will occur to persons of the art. The scope of the invention is limited only by the following claims.

1. A method for enabling a user of a set of headphones, to conduct a self-administered hearing test, wherein said method comprising:
providing a set of headphones;
providing a sound generating module;
providing a controller operative to control separately sounds reaching each one of the user's ears;
providing at least one processor operative to process results obtained from the user of the set of headphones;
providing a user interface coupled to the set of headphones, which enable receiving feedbacks from the user;
loading executable instructions onto the at least one processor to enable performing steps of the self-administered hearing test;
activating the controller and the sound generating module in accordance with the executable instructions, for conducting the self-administered hearing test by the user using the set of headphones;
receiving feedbacks from the user while conducting the self-administered hearing test; and
determining hearing capabilities associated with the user, based on results obtained from the self-administered hearing test.

2. The method of claim 1, wherein said sound generating module, the controller and the at least one processor are all comprised within the set of headphones.

3. The method of claim 1, further comprising a step of processing sounds generated by the sound generating module and automatically adjusting volume and/or amplitude of the audio signals generated by the sound generating module.

4. The method of claim 1, further comprising a step of selectively controlling conveyance of sounds towards one of the user's ears by the controller.

5. The method of claim 1, further comprising a step of preventing audio leakage of sounds from one side of the set of headphones to the other.

6. The method of claim 1, further comprising a step of storing information concerning the hearing capabilities associated with the user retrieved from the results obtained in the self-administered hearing test, and modifying input audio signals in accordance with the stored information, thereby enabling the user to listen to modified audio signals, being the input audio signals after they have been changed into a form that takes into account the user's hearing capabilities.

7. The method of claim 1, wherein the step of determining hearing capabilities associated with the user, is further based on a pre-defined ambient noise.

8. The method of claim 1, wherein the step of determining hearing capabilities associated with the user, comprises establishing a reference model for the hearing capabilities of the user, based on the results obtained from his/her self-administered hearing test.

9. The method of claim 1, wherein the method further comprises a step of providing at least one microphone for detecting one or more current ambient noises and the step of determining hearing capabilities associated with the user comprises taking the one or more current ambient noises as detected by the at least one microphone, into account.

10. The method of claim 1, further comprising a step of generating a constant tone at a pre-defined frequency spectrum and a pre-defined amplitude range, for use while determining hearing capabilities associated with the user.

11. The method of claim 1, further comprising the steps of:
(i) selecting a first frequency band and a respective initial intensity for generating audio tones at said first frequency band;
(ii) determining a user's hearing threshold associated with the selected first frequency band, by generating audio tones at the selected first frequency band at different intensities, starting from said respective initial intensity selected for first frequency band, and changing intensity of the following audio tones generated, until a user's respective hearing threshold for said first frequency band is determined based on one or more feedbacks received from the user;
(iii) selecting another frequency band and a respective initial intensity for generating audio tones at that other frequency band, wherein said respective initial intensity for generating audio tones at that other frequency band is higher than the user's hearing threshold determined for an already tested frequency band;
(iv) determining a hearing threshold of the user in the selected other frequency band, by generating audio tones at the selected other frequency band at different intensities, starting from a respective initial intensity associated with said other frequency band, and changing the intensity of the following audio tones until a user's respective hearing threshold for the other frequency band is determined, based on one or more feedbacks received from the user; and
(v) repeating steps (iii) and (iv) for different frequency bands, until completing the self-administered hearing test.

12. The method of claim 11, wherein the first frequency band is associated with a frequency that is higher than at least one of the other frequency bands and is lower than at least one of the other frequency bands.

13. The method of claim 11, wherein the respective initial intensity for generating audio tones at one of the other frequency bands is higher than the user's hearing threshold determined for an already tested frequency band, by a pre-defined difference.

14. The method of claim 13, wherein the pre-defined difference has a constant value for all other frequency bands tested in said self-administered hearing test.

15. A set of headphones configured to enable a user of the set of headphones to conduct a self-administered hearing test, wherein said set of headphones comprises:
a sound generating module;
a controller operative to control separately sounds reaching each one of the user's ears;
at least one processor operative to process feedbacks received via a user interface coupled to the set of headphones, and wherein the at least one processor is operative to:
allow uploading of executable instructions to enable performing steps in a self-administered hearing test;
activate the controller and the sound generating module in accordance with the executable instructions, for conducting the self-administered hearing test by the user using the set of headphones;
receive the feedbacks from the user while conducting the self-administered hearing test; and
determine hearing capabilities associated with the user, based on results obtained from the self-administered hearing test.

16. The set of headphones of claim 15, wherein the at least one processor is further configured to process sounds generated by the sound generating module, and to automatically adjust volume and/or amplitude of the sounds generated by the sound generating module.

17. The set of headphones of claim 15, wherein the controller is operative to control selective conveyance of sounds towards one of the user's ears.

18. The set of headphones of claim 15, wherein the controller is operative to prevent audio leakage of sounds from one side of the set of headphones to the other.

19. The set of headphones of claim 15, further comprising a memory for storing information concerning the hearing capabilities associated with the user retrieved from the results obtained in the self-administered hearing test, and wherein the at least one processor is further operative to modify incoming audio signals in accordance with the stored information, thereby enabling the user to listen to modified audio signals, being the incoming audio signals after their change into a form that takes into account the user's hearing capabilities.

20. The set of headphones of claim 15, wherein the at least one processor is configured to determine hearing capabilities associated with the user, based on a pre-defined ambient noise.

21. The set of headphones of claim 15, wherein the at least one processor is configured to determine hearing capabilities associated with the user, by establishing a reference model for the hearing capabilities of the user, based on the results obtained from his/her self-administered hearing test.

22. The set of headphones of claim 15, further comprising at least one microphone for detecting current ambient noise and wherein the at least one processor is configured to determine hearing capabilities associated with the user based on the current ambient noise as detected by the at least one microphone.

23. The set of headphones of claim 15, wherein the sound generating module is further configured to generate a constant tone at a pre-defined frequency and a pre-defined amplitude, for use by the at least one processor in determining hearing capabilities associated with the user.

24. A computer program product encoding a computer program stored on a non-transitory computer-readable medium for executing a set of instructions by one or more computer processors for establishing a process for carrying out the steps:
uploading executable instructions to enable performing a self-administered hearing test;
activating a controller and a sound generating module comprised within a set of headphones in accordance with the executable instructions, for conducting the self-administered hearing test by a user using the set of headphones;
receiving feedbacks from the user while conducting the self-administered hearing test; and
determining hearing capabilities associated with the user, based on results obtained from the self-administered hearing test.

25. The computer program product of claim 24, wherein said process further comprises the steps of:
(i) selecting a first frequency band and a respective initial intensity for generating audio tones at said first frequency band;
(ii) determining a user's hearing threshold associated with the selected first frequency band, by generating audio tones at the selected first frequency band at different intensities, starting from said respective initial intensity selected for first frequency band, and changing intensity of the following audio tones generated, until a user's respective hearing threshold for said first frequency band is determined based on one or more feedbacks received from the user;
(iii) selecting another frequency band and a respective initial intensity for generating audio tones at that other frequency band, wherein said respective initial intensity for generating audio tones at that other frequency band is higher than the user's hearing threshold determined for an already tested frequency band;
(iv) determining a hearing threshold of the user in the selected other frequency band, by generating audio tones at the selected other frequency band at different intensities, starting from a respective initial intensity associated with said other frequency band, and changing the intensity of the following audio tones until a user's respective hearing threshold for the other frequency band is determined, based on one or more feedbacks received from the user; and
(v) repeating steps (iii) and (iv) for different frequency bands, until completing the self-administered hearing test.
